(19) United States
(12) Patent Application Publication
(10) Pub. No.: US 2023/0284954 A1
(43) Pub. Date: Sep. 14, 2023

(54) ELECTROCARDIOGRAPHY RESTORATION BY OPERATIONAL CYCLE-GENERATIVE ADVERSARIAL NETWORKS

(71) Applicant: QATAR UNIVERSITY, Doha (QA)

(72) Inventors: Serkan KIRANYAZ, Doha (QA); Ozer Can DEVECIOGLU, Tampere (FI); Turker INCE, Izmir (TR); Junaid MALIK, Tampere (FI); Muhammad CHOWDHURY, Doha (QA); Amith KHANDAKAR, Doha (QA); Moncef GABBOUJ, Tampere (FI); Anas TAHIR, Doha (QA); Tawsifur RAHMAN, Doha (QA)

(21) Appl. No.: 18/119,659

(22) Filed: Mar. 9, 2023

Related U.S. Application Data
(60) Provisional application No. 63/318,153, filed on Mar. 9, 2022.

(51) Int. Cl. A61B 5/28; A61B 8/08

(52) U.S. Cl. CPC A61B 5/28; A61B 8/0833

(57) ABSTRACT

Systems, methods, apparatuses, and computer program products for real-time, personalized cardiac monitoring for early detection of heart-beat anomalies. One method may include a device selecting at least one set of clean ECG segments, and at least one set of corrupted ECG segments; transforming at least one of a one-dimensional or two-dimensional version cycle-CANs trained to transform ECG signals from at least one different dataset; and restoring the at least one set of corrupted ECG segments based upon a one- or two-dimensional operational cycle-GAN trained over the batches.

### CROSS REFERENCE TO RELATED APPLICATION

This application claims the benefit of U.S. Provisional Application No. 63/318,153, filed on Mar. 9, 2022. The entire content of the above-referenced application is hereby incorporated by reference.

### TECHNICAL FIELD

Some example embodiments may generally relate to electrocardiograms (ECGs). For example, certain example embodiments may relate to systems and/or methods for real-time, personalized cardiac monitoring for early detection of heartbeat anomalies.

### BACKGROUND

One of the challenges in collecting and analyzing biomedical signals (e.g., ECG, electroencephalogram (EEG), electrooculogram (EOG), and gait rhythm (GR)) is biomedical data corruption. For example, a Holter monitor is an example of a portable ECG device that may be worn by a patient to monitor cardiac activity over a period of time (e.g., 12 hours, 24 hours). This allows for detecting and recording of sporadic cardiac arrhythmias (e.g., supraventricular tachycardia, ventricular arrhythmia, bradyarrhythmia, palpitations) that could not otherwise be performed within a shorter time period (e.g., during a doctor visit).

### BRIEF DESCRIPTION OF THE DRAWINGS

For a proper understanding of example embodiments, reference should be made to the accompanying drawings, wherein:

FIG. 1a illustrates an example of a 10-second segment of an ECG;

FIG. 1b illustrates another example of a 10-second segment of an ECG;

FIG. 1c illustrates another example of a 10-second segment of an ECG;

FIG. 1d illustrates another example of a 10-second segment of an ECG;

FIG. 2a illustrates another example of a 10-second Holter ECG segment;

FIG. 2b illustrates a GAN reconstructed signal corresponding with the Holter ECG segment of FIG. 2a;

FIG. 2c illustrates another example of a 10-second Holter ECG segment;

FIG. 2d illustrates an operational GAN reconstructed signal (iteration 1) corresponding with the Holter ECG segment of FIG. 2c;

FIG. 2e illustrates an operational GAN reconstructed signal (iteration 2) corresponding with the Holter ECG segment of FIG. 2c;

FIG. 2f illustrates another example of a 10-second Holter ECG segment;

FIG. 2g illustrates an operational GAN reconstructed signal (iteration 1) corresponding with the Holter ECG segment of FIG. 2f;

FIG. 2h illustrates an operational GAN reconstructed signal (iteration 2) corresponding with the Holter ECG segment of FIG. 2f;

FIG. 3 illustrates an example of a flow diagram of a method according to various example embodiments; and

FIG. 4 illustrates an example of various network devices according to some example embodiments.

### DETAILED DESCRIPTION

It will be readily understood that the components of certain example embodiments, as generally described and illustrated in the figures herein, may be arranged and designed in a wide variety of different configurations. Thus, the following detailed description of some example embodiments of systems, methods, apparatuses, and computer program products for real-time, personalized cardiac monitoring for early detection of heartbeat anomalies is not intended to limit the scope of certain example embodiments, but is instead representative of selected example embodiments.

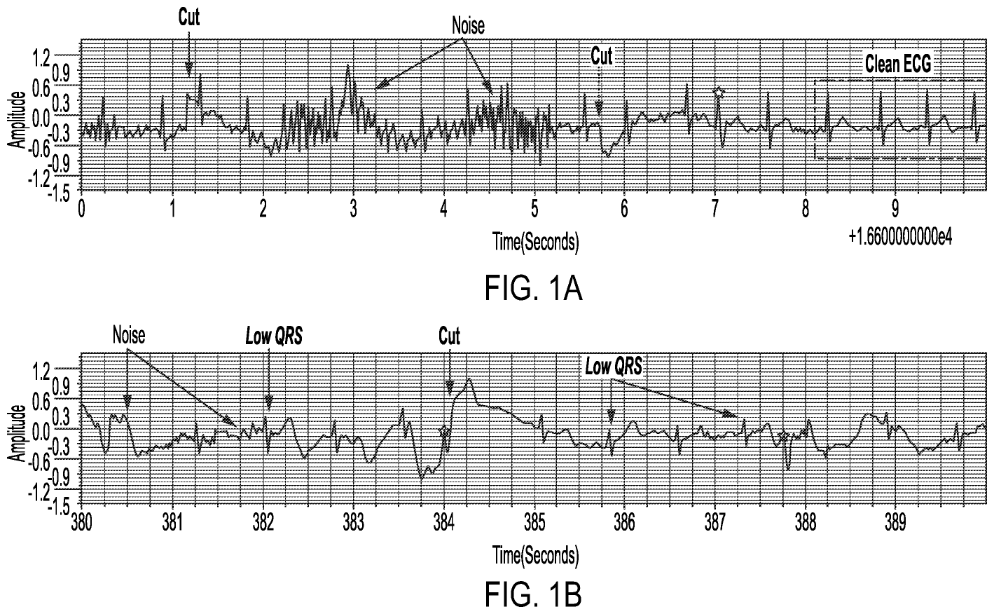

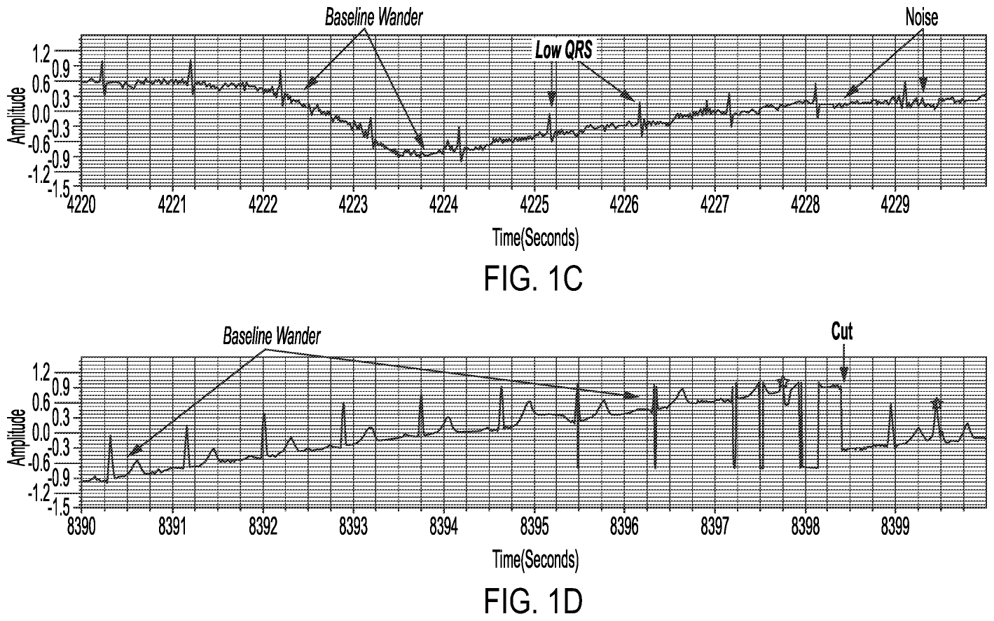

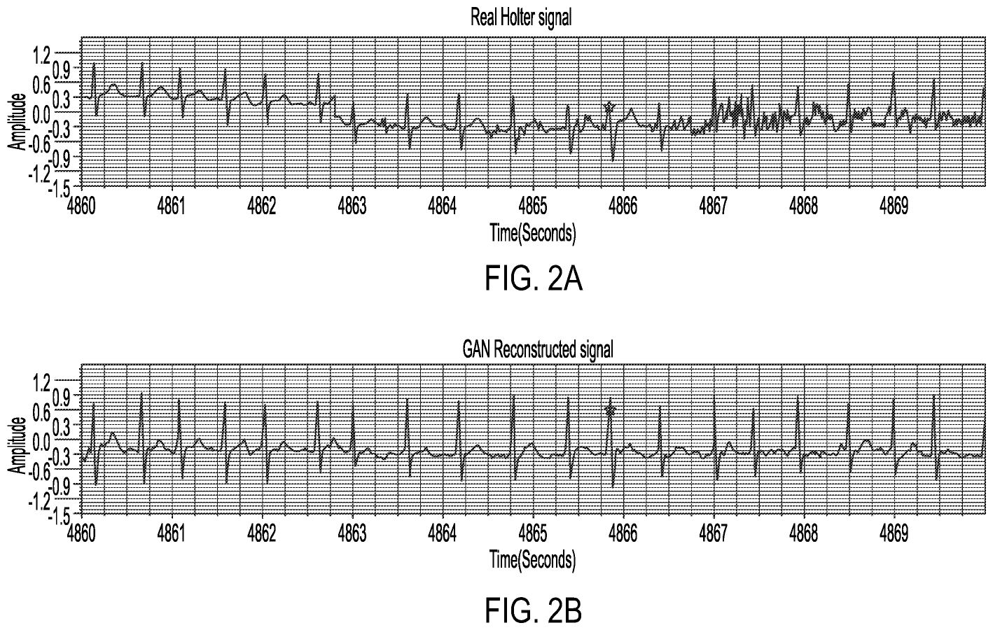

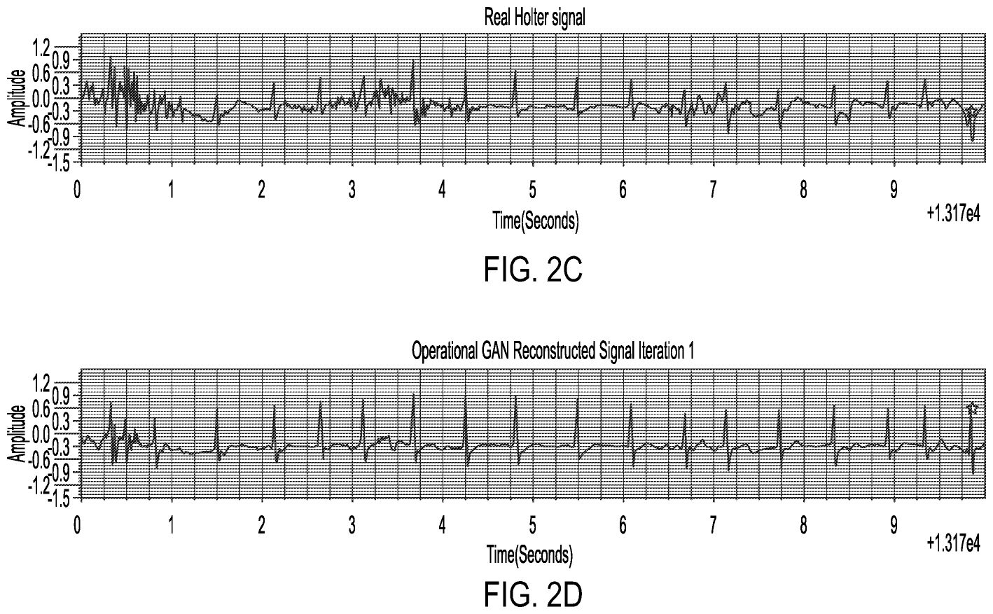

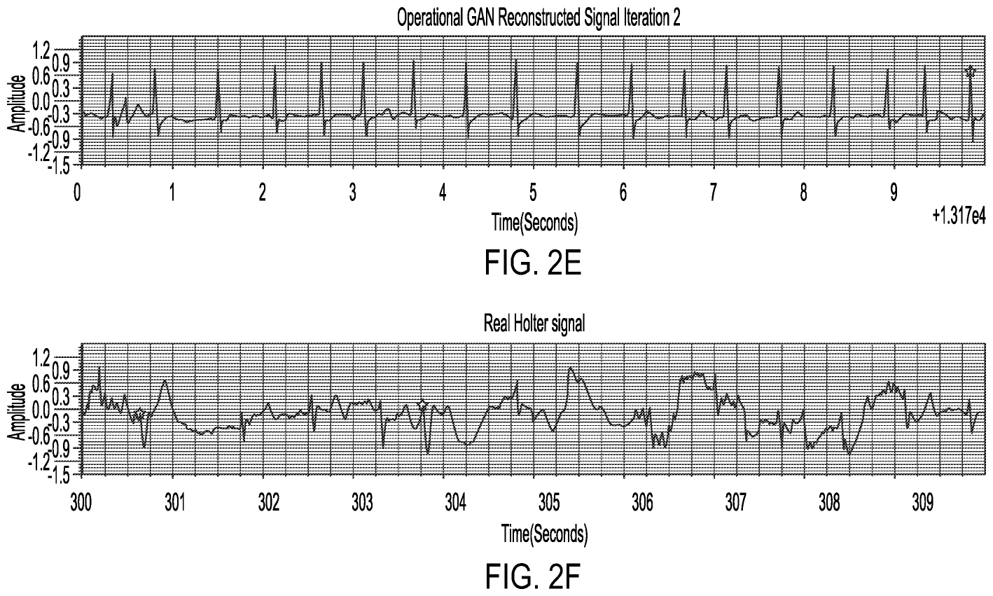

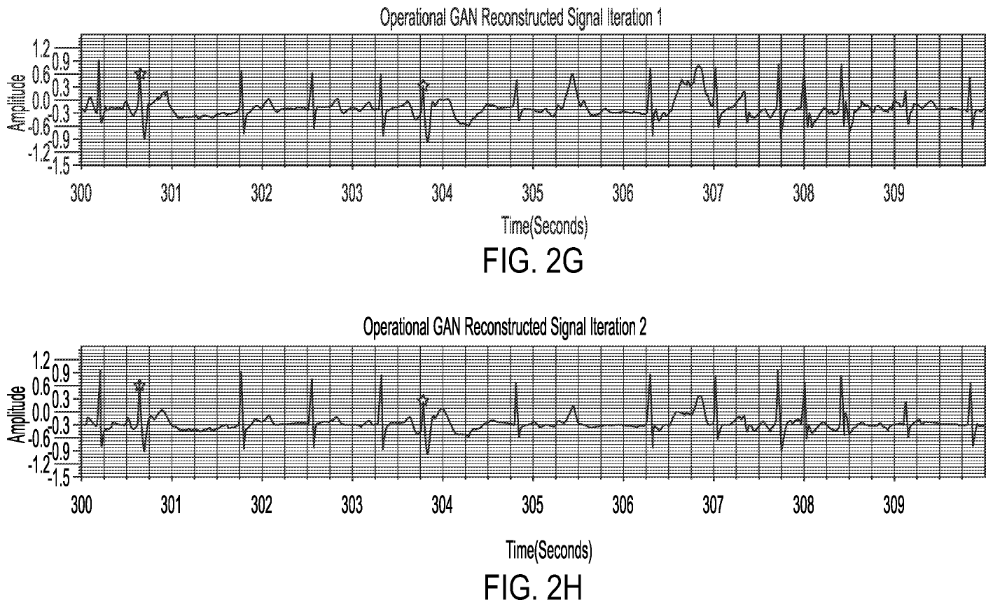

When wearing a portable ECG device, such as a Holter monitor, patients should avoid sudden movements, such as high-impact exercise (e.g., running, basketball) to minimize faulty readings caused by the patient's motion. However, even if such sudden movements are avoided, motion-related slips of the sensor and/or other interference may still cause significant artifacts in the collected data, such as baseline wander, signal cuts, motion artifacts, diminished QRS amplitude, noise, and other interferences. For example, FIGS. 1a-d depict examples of such corrupted ECG recordings applied to datasets from the 2020 China Physiological Signal Challenge (CPSC). In particular, FIGS. 1a-d show a variety of noise, cuts, baseline wander, and low QRS artifacts, where the severity of such blended artifacts can result in the ECG signals being undiagnosable by machines or even experienced doctors. The noise shown in FIGS. 1a-c may be addressed as a “denoising” problem, wherein a certain type of noise may be independent from the signal (e.g., additive Gaussian).

In general, corrupted biomedical signals must be corrected before any accurate analysis can be performed by doctors or machines. Supervised machine learning (ML)-based denoising solutions may be fed a clean signal that has been corrupted by artificial (i.e., additive) noise of a fixed type and variance, and then used as the input. However, this regression problem would not correct the biomedical signals corrupted with a blend of artifacts, as shown in FIGS. 1a-d. For example, in the ECG segment at the first row of FIGS. 1a-d, the noise level may significantly vary within only a few seconds, and is neither additive nor independent from the signal.

Certain example embodiments described herein may have various benefits and/or advantages to overcome the disadvantages described above. For example, certain example embodiments may improve the quality of ECG signals for ECG-related diagnoses, such as arrhythmia classification and peak detection. ECG restoration may be performed, where a corrupted ECG signal can be recovered with a clinical quality level. Rather than applying previous “denoising” solutions for additive (i.e., artificial) noise with a fixed type and power, some example embodiments may use a blind restoration approach without any prior assumption over the artifact types and severity. Furthermore, various example embodiments may be fully automatic, and can be automatically applied to the ECG signal without any tuning or pre-processing. In addition to providing superior ECG quality, hidden/undetected arrhythmia events can be diagnosed from the restored ECG. Certain example embodiments may also provide a real-time solution for low-power mobile devices since there is no requirement for pre- or post-processing and manual feature-extraction operations. Thus, certain example embodiments discussed below are directed to improvements in computer-related technology.

Certain example embodiments described herein may be used in personal ECG monitors, Holter registers, mobile devices with ECG acquisition capabilities (e.g., single-lead), and dedicated PC applications as the default ECG restoration module.

As used in some example embodiments, GANs, cycle-consistent adversarial networks (Cycle-GANs), and their variations may provide image-to-image translation on unpaired datasets. Since cycle-GANs can preserve major “patterns” of the signal transformed to the “other” category when a corrupted ECG segment is transformed to a clean segment, the main ECG characteristics (e.g., the interval and timing of R-peaks, QRS waveform of ECG beats, etc.) may be preserved while improving the quality. In order to further boost the restoration performance and reduce the complexity, operational cycle-GANs may be used. Derived from generalized operational perceptrons, operational neural networks (ONNs), and their new variants, self-organized operational neural networks (Self-ONNs), are heterogeneous network models with a non-linear neuron model. Self-ONNs may be heterogeneous network models with a non-linear neuron model which have shown superior diversity and increased learning capabilities. Such self-ONNs may outperform CNNs in many regression and classification tasks.

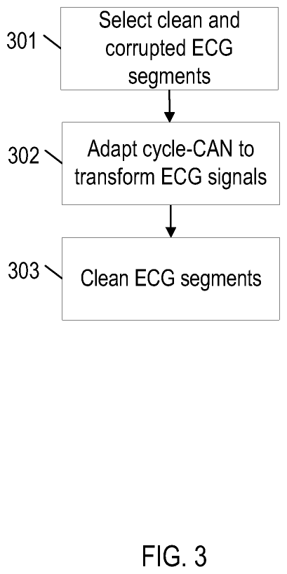

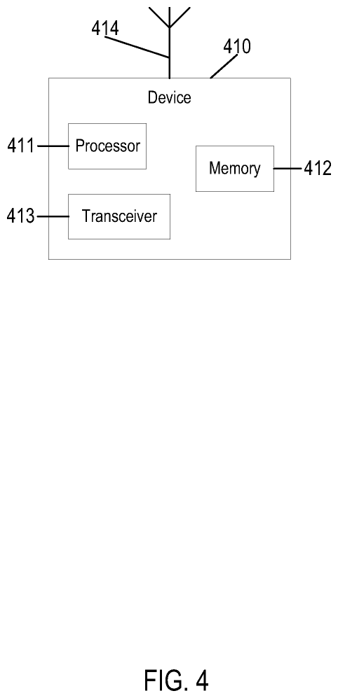

FIG. 3 illustrates an example of a flow diagram of a method that may be performed by a device, such as device 410 illustrated in FIG. 4, according to various example embodiments.

At step 301, the method may include selecting at least one set of clean ECG segments, and at least one set of corrupted ECG segments, such as from a dataset.

At step 302, the method may include adapting at least one of a one-dimensional or two-dimensional version cycle-consistent adversarial networks trained to transform ECG signals (i.e., segments) from at least one different dataset. As an example, the transformed ECG signals may be used as a baseline.

In various example embodiments, the convolutional layers/neurons of the native cycle-GANs may be replaced by operational/generative layers/neurons of self-ONNs.

At step 303, once a one- or two-dimensional operational cycle-GAN is trained over the batches, the generator self-ONN trained for the “corrupted” to “clean” ECG segment transformation can then be used for the ECG restoration.

FIG. 4 illustrates an example of a system according to certain example embodiments. In one example embodiment, a system may include multiple devices, such as, for example, device 410.

Device 410 may include one or more of a stationary, mobile, or ambulatory ECG, a mobile device, such as a mobile phone, smart phone, personal digital assistant (PDA), tablet, or portable media player, digital camera, pocket video camera, video game console, navigation unit, such as a global positioning system (GPS) device, desktop or laptop computer, single-location device, such as a sensor or smart meter, or any combination thereof. Furthermore, device 410 may be one or more of a citizens broadband radio service device (CBSD).

Device 410 may include at least one processor, indicated as 411. Processor 411 may be embodied by any computational or data processing device, such as a central processing unit (CPU), application specific integrated circuit (ASIC), or comparable device. The processors may be implemented as a single controller, or a plurality of controllers or processors.

At least one memory may be provided in one or more of the devices, as indicated at 412. The memory may be fixed or removable. The memory may include computer program instructions or computer code contained therein. Memory 412 may independently be any suitable storage device, such as a non-transitory computer-readable medium. The term “non-transitory,” as used herein, may correspond to a limitation of the medium itself (i.e., tangible, not a signal) as opposed to a limitation on data storage persistency (e.g., random access memory (RAM) vs. read-only memory (ROM)). A hard disk drive (HDD), random access memory (RAM), flash memory, or other suitable memory may be used. The memories may be combined on a single integrated circuit as the processor, or may be separate from the one or more processors. Furthermore, the computer program instructions stored in the memory, and which may be processed by the processors, may be any suitable form of computer program code, for example, a compiled or interpreted computer program written in any suitable programming language.

Processor 411 and memory 412, and any subset thereof, may be configured to provide means corresponding to the various blocks of FIG. 3. Although not shown, the devices may also include positioning hardware, such as GPS or micro electrical mechanical system (MEMS) hardware, which may be used to determine a location of the device. Other sensors are also permitted, and may be configured to determine location, elevation, velocity, orientation, and so forth, such as barometers, compasses, and the like.

As shown in FIG. 4, transceiver 413 may be provided, and one or more devices may also include at least one antenna, illustrated as 414. The device may have many antennas, such as an array of antennas configured for multiple input multiple output (MIMO) communications, or multiple antennas for multiple RATs. Other configurations of these devices, for example, may be provided. Transceiver 413 may be a transmitter, a receiver, both a transmitter and a receiver, or a unit or device that may be configured both for transmission and reception.

The memory and the computer program instructions may be configured, with the processor for the particular device, to cause a hardware apparatus, such as UE, to perform any of the processes described above (i.e., FIG. 3). Therefore, in certain example embodiments, a non-transitory computer-readable medium may be encoded with computer instructions that, when executed in hardware, perform a process such as one of the processes described herein. Alternatively, certain example embodiments may be performed entirely in hardware.

In certain example embodiments, an apparatus may include circuitry configured to perform any of the processes or functions illustrated in FIG. 3. As used in this application, the term “circuitry” may refer to one or more or all of the following: (a) hardware-only circuit implementations (such as implementations in only analog and/or digital circuitry), (b) combinations of hardware circuits and software, such as (as applicable): (i) a combination of analog and/or digital hardware circuit(s) with software/firmware and (ii) any portions of hardware processor(s) with software (including digital signal processor(s)), software, and memory(ies) that work together to cause an apparatus, such as a mobile phone or server, to perform various functions), and (c) hardware circuit(s) and or processor(s), such as a microprocessor(s) or a portion of a microprocessor(s), that requires software (e.g., firmware) for operation, but the software may not be present when it is not needed for operation. This definition of circuitry applies to all uses of this term in this application, including in any claims. As a further example, as used in this application, the term circuitry also covers an implementation of merely a hardware circuit or processor (or multiple processors) or portion of a hardware circuit or processor and its (or their) accompanying software and/or firmware. The term circuitry also covers, for example and if applicable to the particular claim element, a baseband integrated circuit or processor integrated circuit for a mobile device or a similar integrated circuit in server, a cellular network device, or other computing or network device.

According to certain example embodiments, processor 411, and memory 412, may be included in or may form a part of processing circuitry or control circuitry. In addition, in some example embodiments, transceiver 413 may be included in or may form a part of transceiving circuitry.

In some example embodiments, an apparatus (e.g., device 410) may include means for performing a method, a process, or any of the variants discussed herein. Examples of the means may include one or more processors, memory, controllers, transmitters, receivers, and/or computer program code for causing the performance of the operations.

The features, structures, or characteristics of example embodiments described throughout this specification may be combined in any suitable manner in one or more example embodiments. For example, the usage of the phrases “various embodiments,” “certain embodiments,” “some embodiments,” or other similar language throughout this specification refers to the fact that a particular feature, structure, or characteristic described in connection with an example embodiment may be included in at least one example embodiment. Thus, appearances of the phrases “in various embodiments,” “in certain embodiments,” “in some embodiments,” or other similar language throughout this specification does not necessarily all refer to the same group of example embodiments, and the described features, structures, or characteristics may be combined in any suitable manner in one or more example embodiments.

As used herein, “at least one of the following: <a list of two or more elements>” and “at least one of <a list of two or more elements>” and similar wording, where the list of two or more elements are joined by “and” or “or,” mean at least any one of the elements, or at least any two or more of the elements, or at least all the elements.

Additionally, if desired, the different functions or procedures discussed above may be performed in a different order and/or concurrently with each other. Furthermore, if desired, one or more of the described functions or procedures may be optional or may be combined. As such, the description above should be considered as illustrative of the principles and teachings of certain example embodiments, and not in limitation thereof.

One having ordinary skill in the art will readily understand that the example embodiments discussed above may be practiced with procedures in a different order, and/or with hardware elements in configurations which are different than those which are disclosed. Therefore, although some embodiments have been described based upon these example embodiments, it would be apparent to those of skill in the art that certain modifications, variations, and alternative constructions would be apparent, while remaining within the spirit and scope of the example embodiments.

### Partial Glossary

CNN Convolutional Neural Network
CPSC China Physiological Signal Challenge
ECG Electrocardiography
GAN Generative Adversarial Network
ML Machine Learning
ONN Operational Neural Network
PC Personal Computer
QRS Q, R, and S wave

We claim:

1. A method comprising:
selecting, by a device, at least one set of clean electrocardiogram segments, and at least one set of corrupted electrocardiogram segments;
transforming, by the device, at least one of a one-dimensional or two-dimensional version cycle-consistent adversarial networks trained to transform electrocardiogram signals from at least one different dataset; and
restoring, by the device, the at least one set of corrupted electrocardiogram segments based upon a one- or two-dimensional operational cycle-generative adversarial network trained over the batches.

2. An apparatus comprising:
at least one processor; and
at least one memory storing instructions that, when executed by the at least one processor, cause the apparatus at least to:
selecting, by a device, at least one set of clean electrocardiogram segments, and at least one set of corrupted electrocardiogram segments;
transforming, by the device, at least one of a one-dimensional or two-dimensional version cycle-consistent adversarial networks trained to transform electrocardiogram signals from at least one different dataset; and
restoring, by the device, the at least one set of corrupted electrocardiogram segments based upon a one- or two-dimensional operational cycle-generative adversarial network trained over the batches.
